(19) United States
(12) Patent Application Publication
(10) Pub. No.: US 2025/0098798 A1
(43) Pub. Date: Mar. 27, 2025

(54) VIRUS/BIOHAZARD INDICATING DISPOSABLE FACE MASK

(71) Applicant: Keith H. TAYLOR, Farmington Hills, MI (US)

(72) Inventor: Keith H. TAYLOR, Farmington Hills, MI (US)

(21) Appl. No.: 18/728,153

(22) PCT Filed: Jan. 11, 2023
(86) PCT No.: PCT/US23/60454

§ 371 Date: Jul. 11, 2024

Related U.S. Application Data
(60) Provisional application No. 63/300,118, filed on Jan. 17, 2022.

(51) Int. Cl. A41D 13/00; A41D 13/11

(52) U.S. Cl. CPC A41D 13/0002; A41D 13/0012; A41D 13/1161; A41D 2400/52

(57) ABSTRACT

A face mask, including a body and at least one securing mechanism operatively attached thereto, and a used indicator mechanism for indicating that the face mask is used and an infectious disease detection mechanism for indicating if the user wearing the face mask has an infectious disease operatively integrated into the body. A method of indicating whether a face mask is used, by a user actuating a used indicator mechanism on the face mask, wearing the face mask, and the used indicator mechanism indicating that the face mask is used. A method of indicating whether a user has an infectious disease, by a user actuating an infectious disease mechanism on a face mask, wearing the face mask, and the infectious disease mechanism indicating that the user has an infectious disease by changing from a first color to a second color.

### BACKGROUND OF THE INVENTION

### 1. Technical Field

The present invention relates to compositions and methods for detecting viruses and other biohazards. More specifically, the present invention relates to face masks that indicate the presence of a virus or biohazard.

### 2. Background Art

Infectious diseases are diseases caused by pathogenic microorganisms such as bacteria, viruses, parasites, or fungi that can be spread from person to person, either directly or indirectly.

Coronavirus Disease 2019 (COVID-19) is a severe acute respiratory syndrome (SARS) coronavirus 2 that originated in 2019 in Wuhan, China, and has quickly spread around the world. The viral infection is spread from person to person by respiratory droplets. Symptoms include fever, cough, shortness of breath, loss of smell and taste, and it is very similar to influenza. Due to the presence of infected asymptomatic individuals, it is hard to tell who is actually infected. While many people recover and have mild symptoms, those who are older and have comorbidities are at risk of serious illness or death. While tests are available to identify COVID-19, there is a delay in test results due to the overwhelming request for tests and individuals could be infected and continue spreading the virus. Individuals who have been identified as having the virus need to quarantine themselves but cannot do this if they cannot receive test results quickly. Furthermore, even if an individual tests negative, this is not necessarily indicative of their current state of health, which could foster a false consensus that once having undergone testing, it is fine to mix and mingle as they used to with others.

It has been determined that wearing a face covering or face mask can greatly reduce the spread of COVID-19, especially since one cannot necessarily tell if they are infected. A study by Chu, et al. (The Lancet, Jun. 1, 2021) reports that with the use of face masks, the chance of transmission of COVID-19 was 3% as compared to 17% without a mask. However, many individuals fail to use masks with inconsistent messages from the government, and many use them incorrectly.

Therefore, there remains a need for methods of indicating infected individuals with COVID-19 as well as other infectious diseases, as well as improved face masks.

### SUMMARY OF THE INVENTION

The present invention provides for a face mask, including a body and at least one securing mechanism operatively attached thereto, and a used indicator mechanism for indicating that the face mask is used and an infectious disease detection mechanism for indicating if the user wearing the face mask has an infectious disease operatively integrated into the body.

The present invention provides for a method of indicating whether a face mask is used, by a user actuating a used indicator mechanism on the face mask, wearing the face mask, and the used indicator mechanism indicating that the face mask is used.

The present invention also provides for a method of indicating whether a user has an infectious disease, by a user actuating an infectious disease mechanism on a face mask, wearing the face mask, and the infectious disease mechanism indicating that the user has an infectious disease by changing from a first color to a second color.

### DESCRIPTION OF THE DRAWINGS

Other advantages of the present invention are readily appreciated as the same becomes better understood by reference to the following detailed description when considered in connection with the accompanying drawings wherein:

FIG. 1 is a top view of a face mask;

FIG. 2 is a top view of a face mask with a used indicator mechanism/virus detection mechanism activated; and

FIG. 3 is a back view of the face mask.

### DETAILED DESCRIPTION OF THE INVENTION

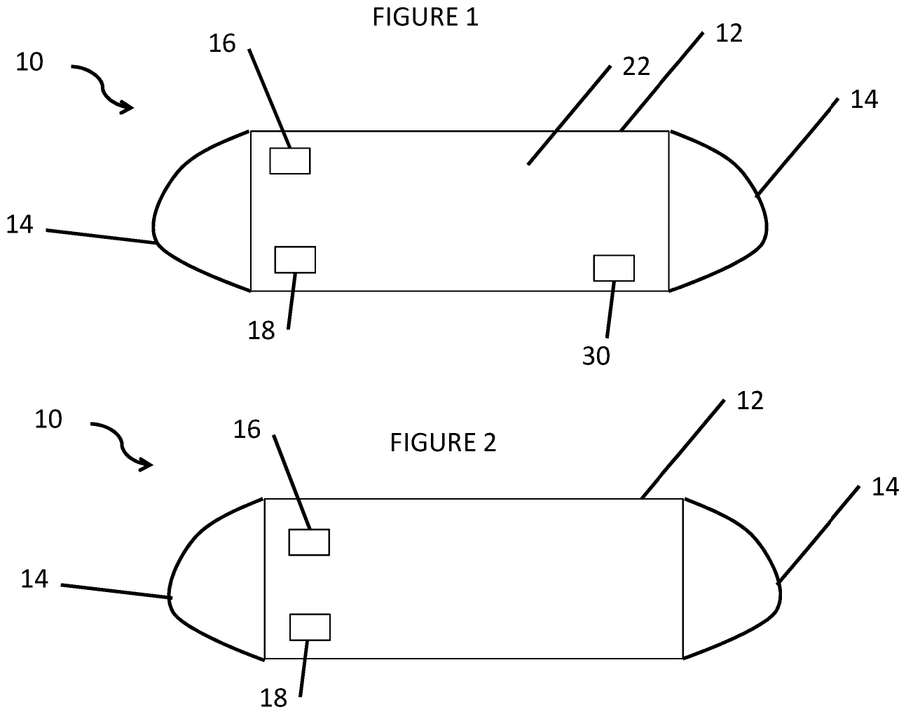

The present invention provides for a face mask 10, shown in FIGS. 1 and 2, including a body 12 and at least one securing mechanism 14 operatively attached thereto, and a used indicator mechanism 16 for indicating that the face mask 10 is used and an infectious disease detection mechanism 18 for indicating if the user wearing the face mask 10 has an infectious disease operatively integrated into the body 12.

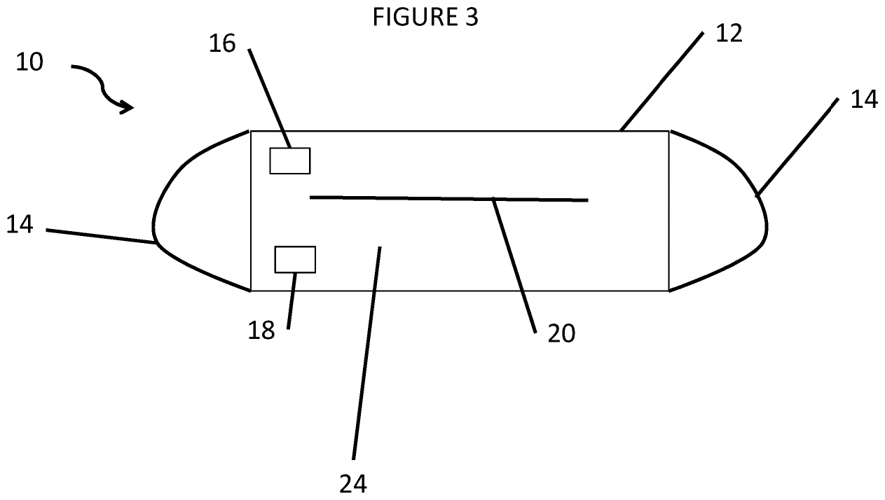

The body 12 can be made of any suitable material, such as any cloth, cotton, nylon, or filter material and can include multiple layers. The body 12 can be in different sizes to fit children (i.e., child sized) or adult (i.e., adult sized) faces. The body 12 can include a pocket 20 for filters on an inner surface 24 (shown in FIG. 3).

The face mask 10 is secured on an user's head with the securing mechanism 14 (such as elastic ear loops attached at opposite ends of the body 12 shown in FIGS. 1 and 2). Other designs can include loops or ties that fit around the head, ties that tie around the ears, or combinations of loops and ties.

The used indicator mechanism 16 indicates whether the face mask 10 has been used and whether it should be disposed of. The used indicator mechanism 16 can emit an odor after 24 hours of use of the face mask 10 (or other appropriate predetermined periods of time, such as 1 hour, 5 hours, 10 hours, 15 hours, or 20 hours). If the face mask 10 smells, this is an indication to the user not to put the face mask 10 on because it is used, or to take it off because it has been used too long. The used indicator mechanism 16 can alternatively, or in addition, be a color change mechanism wherein the body 12 changes from a first unused color (FIG. 1) to a second used color (FIG. 2). The color change mechanism can be a solid color or a colored design. Any distinguishable colors can be used, such as, but not limited to, red, orange, yellow, green, blue, purple, grey, black, white. The used indicator mechanism 16 can be passively activated (such as the user putting on the face mask 10) or actively activated (such as by the user coughing into the face mask 10, clicking an actuator on the face mask 10, opening packaging containing the face mask 10, peeling off a layer protecting the used indicator mechanism 16, or any other suitable method). The used indicator mechanism 16 can be integrated into the body 12, either the entire body 12 or a portion of the body 12, and be on an outer surface 22 of the body, an inner surface 24 of the body, or both.

The infectious disease detection mechanism 18 indicates whether the user has an infectious disease. The infectious disease detection mechanism 18 can include any suitable detection mechanism to indicate the presence of disease, such as antibody detection, DNA detection, RNA detection, or protein detection.

The infectious disease detection mechanism 18 includes a color change mechanism wherein the body 12 changes from a first color (FIG. 1) indicating no disease to a second color (FIG. 2) indicating the presence of disease. The color change mechanism can be a solid color or a colored design. Any distinguishable colors can be used, such as, but not limited to, red, orange, yellow, green, blue, purple, grey, black, white. Preferably, the color is different than that used for the used indicator mechanism 16. The infectious disease detection mechanism 18 can be passively activated (such as the user putting on the face mask 10) or actively activated (such as by the user coughing into the face mask 10, clicking an actuator on the face mask 10, opening packaging containing the face mask 10, peeling off a layer protecting the infectious disease detection mechanism 18 before use, or any other suitable method). The infectious disease detection mechanism 18 can be integrated into the body 12, either the entire body 12 or a portion of the body 12, and can be on a separate portion of the body 12 than the used indicator mechanism 16. The infectious disease detection mechanism 18 can be on an outer surface 22 of the body 12 (visible to others, which is useful to let other know of exposure to disease), an inner surface 24 of the body 12 (the side against a user's face), or both. The infectious disease detection mechanism 18 can also be integrated on a disposable filter insert inside the pocket 20 in the body 12. The infectious disease detection mechanism 18 can indicate the user's disease status in real time and indicate whether they should seek medical attention.

The outer surface 22 of the body 12 can further include a disease environmental presence indicator 30 operatively integrated thereon in combination with the infectious disease detection mechanism 18 and/or the used indicator mechanism 16, shown in FIG. 1. The disease environmental presence indicator 30 indicates whether there is any disease present in the surrounding environment. This can be helpful to a user so that they can leave a particular place, such as a store, school, office, etc. when the presence of disease is detected. The disease environmental presence indicator 30 can be a color change mechanism as described above, and can change a different color than the color of the face mask 10, a light activated by the presence of the disease, or any other indicating mechanism. When the disease environmental presence indicator 30 is included, the face mask 10 can include a membrane or coating between the outer surface 22 and inner surface 24 so that the disease environmental presence indicator 30 is not set off by the user and vice versa. The membrane/coating can be a microporous membrane made from polymers of polytetrafluoroethylene (PTFE), polyurethane, polyolefins, polyamides, polyester, polyether, or polyether based copolymers. Pore sizes can range 0.03 micrometers to 10 micrometers in diameter for example. The membrane or coating can allow the user to breath but not let disease molecules in or out of the face mask 10.

The infectious disease can include a viral, protozoan, or bacterial disease such as most preferably influenza, measles, or COVID-19, or any of AIDS, amebiasis, anaplasmosis, anthrax, antibiotic resistance, avian influenza, babesiosis, botulism, brucellosis, campylobacter, cat scratch disease, chickenpox, chikungunya, Chlamydia trachomatis, cholera, Clostridium perfringens, conjunctivitis, crusted scabies, cryptosporidiosis, cyclospora, dengue fever, diphtheria, Ebola virus disease, E. coli, eastern equine encephalitis (EEE), enterovirus 68, fifth disease, genital herpes, genital warts, giardia, gonorrhea, group A Streptococcus, Guillain-Barré syndrome, Hand, Foot & Mouth Disease, Hansen's disease, hantavirus, lice, hepatitis A, hepatitis B, hepatitis C, herpes, herpes B virus, Hib disease, histoplasmosis, HIV, HPV (Human Papillomavirus), impetigo, Kawasaki syndrome, legionellosis, leprosy, leptospirosis, listeriosis, lyme disease, lymphocytic choriomeningitis (LCMV), malaria, Marburg virus, meningitis, meningococcal disease, MERS (Middle East Respiratory Illness), monkeypox, mononucleosis, MRSA, mumps, Mycoplasma pneumoniae, neisseria meningitis, norovirus, Orf Virus (Sore Mouth), pelvic inflammatory disease (PID), PEP, pertussis, pink eye, plague, pneumococcal disease, powassan virus, psittacosis, Q fever, rabies, raccoon roundworm, rat bite fever, Reye's Syndrome, Rickettsialpox, ringworm, rubella, salmonella, scabies, scarlet fever, shigella, shingles, smallpox, strep throat, syphilis, tetanus, toxoplasmosis, trichinosis, trichomoniasis, tuberculosis, tularemia, varicella, vibriosis, viral hemorrhagic fevers (VHF), West Nile virus, whooping cough, yellow fever, yersiniosis, or zika virus.

The present invention provides for a method of indicating whether a face mask 10 is used, by a user actuating a used indicator mechanism 16 on the face mask 10, wearing the face mask 10, and the used indicator mechanism 16 indicating that the face mask 10 is used. Preferably, the used indicator mechanism 16 indicates that the face mask 10 is used through an odor and/or a color change as described above after a set/predetermined amount of time of use, and the user can then dispose of the face mask 10.

The present invention also provides for a method of indicating whether a user has an infectious disease, by a user actuating an infectious disease mechanism 18 on a face mask 10, wearing the face mask 10, and the infectious disease mechanism 18 indicating that the user has an infectious disease by changing from a first color to a second color. By performing this method, the user can either indicate to others that they are disease-free or that they need to seek medical attention or quarantine themselves. The method can further include the step of indicating that there is disease in the environment by the disease environmental presence indicator 30 changing color or by any other indicating mechanism. This can alert the user to leave the place that they are at and move to a safer place. The method can include preventing disease molecules from entering the face mask 10 or exiting the face mask 10 with the membrane or coating.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A face mask comprising a body and at least one securing mechanism operatively attached to said body, and a used indicator mechanism for indicating that said face mask is used and an infectious disease detection mechanism for indicating if a user wearing said face mask has an infectious disease operatively integrated into said body.

2. The face mask of claim 1, wherein said body is made of a material chosen from the group consisting of cloth, cotton, nylon, and filter material.

3. The face mask of claim 1, wherein said body is a size chosen from the group consisting of child sized and adult sized.

4. The face mask of claim 1, wherein said body further includes a pocket for filters.

5. The face mask of claim 1, wherein said securing mechanism is chosen from the group consisting of elastic ear loops, ties, and combinations thereof.

6. The face mask of claim 1, wherein said used indicator mechanism omits an odor after a predetermined time of use of said face mask.

7. The face mask of claim 1, wherein said used indicator mechanism is further defined as a color change mechanism for changing said body from a first unused color to a second used color.

8. The face mask of claim 7, wherein said first unused color is chosen from the group consisting of red, orange, yellow, green, blue, purple, grey, black, and white and wherein said second used color is chosen from the group consisting of red, orange, yellow, green, blue, purple, grey, black, and white.

9. The face mask of claim 1, wherein said used indicator mechanism is activated by a method chosen from the group consisting of passive activation and active activation.

10. The face mask of claim 1, wherein said used indicator mechanism is integrated into an entirety of said body or a portion of said body.

11. The face mask of claim 1, wherein said infectious disease detection mechanism includes a mechanism chosen from the group consisting of antibody detection, DNA detection, RNA detection, and protein detection.

12. The face mask of claim 1, wherein said infectious disease detection mechanism includes a color change mechanism wherein said body changes from a first color indicating no disease to a second color indicating presence of disease.

13. The face mask of claim 12, wherein said color change mechanism is a different color than said used indicator mechanism.

14. The face mask of claim 12, wherein said infectious disease detection mechanism is activated by a method chosen from the group consisting of passive activation and active activation.

15. The face mask of claim 12, wherein said infectious disease detection mechanism is integrated into an entirety of said body or a portion of said body.

16. The face mask of claim 12, wherein said infectious disease detection mechanism indicates said user's disease status in real time.

17. The face mask of claim 12, wherein said infectious disease detection mechanism is located at a part of said body chosen from the group consisting of an outer surface, an inner surface, an outer surface and an inner surface, and integrated on a disposable filter insert inside a pocket in said body.

18. The face mask of claim 1, wherein the infectious disease is chosen from the group consisting of influenza, measles, COVID-19, AIDS, amebiasis, anaplasmosis, anthrax, antibiotic resistance, avian influenza, babesiosis, botulism, brucellosis, campylobacter, cat scratch disease, chickenpox, chikungunya, Chlamydia trachomatis, cholera, Clostridium perfringens, conjunctivitis, crusted scabies, cryptosporidiosis, cyclospora, dengue fever, diphtheria, Ebola virus disease, E. coli, eastern equine encephalitis (EEE), enterovirus 68, fifth disease, genital herpes, genital warts, giardia, gonorrhea, group A Streptococcus, Guillain-Barré syndrome, Hand, Foot & Mouth Disease, Hansen's disease, hantavirus, lice, hepatitis A, hepatitis B, hepatitis C, herpes, herpes B virus, Hib disease, histoplasmosis, HIV, HPV (Human Papillomavirus), impetigo, Kawasaki syndrome, legionellosis, leprosy, leptospirosis, listeriosis, lyme disease, lymphocytic choriomeningitis (LCMV), malaria, Marburg virus, meningitis, meningococcal disease, MERS (Middle East Respiratory Illness), monkeypox, mononucleosis, MRSA, mumps, Mycoplasma pneumoniae, neisseria meningitis, norovirus, Orf Virus (Sore Mouth), pelvic inflammatory disease (PID), PEP, pertussis, pink eye, plague, pneumococcal disease, powassan virus, psittacosis, Q fever, rabies, raccoon roundworm, rat bite fever, Reye's Syndrome, Rickettsialpox, ringworm, rubella, salmonella, scabies, scarlet fever, shigella, shingles, smallpox, strep throat, syphilis, tetanus, toxoplasmosis, trichinosis, trichomoniasis, tuberculosis, tularemia, varicella, vibriosis, viral hemorrhagic fevers (VHF), West Nile virus, whooping cough, yellow fever, yersiniosis, and zika virus.

19. The face mask of claim 1, further including a disease environmental presence indicator operatively integrated thereon for indicating whether there is any disease present in the surrounding environment.

20. The face mask of claim 19, wherein said disease environmental presence indicator is further defined as a mechanism chosen from the group consisting of a color change mechanism or a light activated by presence of disease.

21. The face mask of claim 19, further including a membrane between an outer surface and an inner surface of said face mask.

22. The face mask of claim 21, wherein said membrane is made of a material chosen from the group consisting of polytetrafluoroethylene, polyurethane, polyolefins, polyamides, polyester, polyether, and polyether based copolymers.

23. A method of indicating whether a face mask is used, including the steps of:
a user actuating a used indicator mechanism on a face mask;
wearing the face mask; and
the used indicator mechanism indicating that the face mask is used.

24. The method of claim 23, wherein said indicating step is further defined as the used indicator mechanism performing a step chosen from the group consisting of emitting an odor, the used indicator mechanism changing a body of the face mask from a first color to a second color, and combinations thereof.

25. The method of claim 23, wherein said actuating step is further defined as a step chosen from the group consisting of the user putting on the face mask, the user coughing into the face mask, clicking an actuator on the face mask, opening packaging containing the face mask, and peeling off a layer protecting the used indicator mechanism.

26. A method of indicating whether a user has an infectious disease, including the steps of:
a user actuating an infectious disease mechanism on a face mask;
wearing the face mask; and
the infectious disease mechanism indicating that the user has an infectious disease by changing from a first color to a second color.

27. The method of claim 26, wherein said actuating step is further defined as a step chosen from the group consisting of the user putting on the face mask, the user coughing into the face mask, clicking an actuator on the face mask, opening packaging containing the face mask, and peeling off a layer protecting the infectious disease mechanism.

28. The method of claim 26, wherein the infectious disease is chosen from the group consisting of influenza, measles, COVID-19, AIDS, amebiasis, anaplasmosis, anthrax, antibiotic resistance, avian influenza, babesiosis, botulism, brucellosis, campylobacter, cat scratch disease, chickenpox, chikungunya, Chlamydia trachomatis, cholera, Clostridium perfringens, conjunctivitis, crusted scabies, cryptosporidiosis, cyclospora, dengue fever, diphtheria, Ebola virus disease, E. coli, eastern equine encephalitis (EEE), enterovirus 68, fifth disease, genital herpes, genital warts, giardia, gonorrhea, group A Streptococcus, Guillain-Barré syndrome, Hand, Foot & Mouth Disease, Hansen's disease, hantavirus, lice, hepatitis A, hepatitis B, hepatitis C, herpes, herpes B virus, Hib disease, histoplasmosis, HIV, HPV (Human Papillomavirus), impetigo, Kawasaki syndrome, legionellosis, leprosy, leptospirosis, listeriosis, lyme disease, lymphocytic choriomeningitis (LCMV), malaria, Marburg virus, meningitis, meningococcal disease, MERS (Middle East Respiratory Illness), monkeypox, mononucleosis, MRSA, mumps, Mycoplasma pneumoniae, neisseria meningitis, norovirus, Orf Virus (Sore Mouth), pelvic inflammatory disease (PID), PEP, pertussis, pink eye, plague, pneumococcal disease, powassan virus, psittacosis, Q fever, rabies, raccoon roundworm, rat bite fever, Reye's Syndrome, Rickettsialpox, ringworm, rubella, salmonella, scabies, scarlet fever, shigella, shingles, smallpox, strep throat, syphilis, tetanus, toxoplasmosis, trichinosis, trichomoniasis, tuberculosis, tularemia, varicella, vibriosis, viral hemorrhagic fevers (VHF), West Nile virus, whooping cough, yellow fever, yersiniosis, and zika virus.

29. The method of claim 26, further including the step of indicating that there is disease in the environment.

30. The method of claim 26, further including the step of preventing disease molecules from entering the face mask or exiting the face mask with a membrane between an outer surface and inner surface of the face mask.
